(19) United States
(12) Patent Application Publication
(10) Pub. No.: US 2021/0322197 A1
(43) Pub. Date: Oct. 21, 2021

(54) ORTHOTIC APPLIANCE WITH CONTINUOUSLY ADJUSTABLE POSITIONING OF CORRECTIVE ELEMENTS

(71) Applicant: Motion Therapeutics, Inc., Oakland, CA (US)

(72) Inventors: Cynthia GIBSON-HORN, Oakland, CA (US); David PEARSON, Thousand Oaks, CA (US); Mark SUNDERLAND, Philadelphia, PA (US); Andrew LESHER, Wynnewood, PA (US); Elle ABBEY, Philadelphia, PA (US)

(21) Appl. No.: 17/364,830

(22) Filed: Jun. 30, 2021

Related U.S. Application Data
(63) Continuation-in-part of application No. 15/425,602, filed on Feb. 6, 2017.

(51) Int. Cl. A61F 5/02; A61N 1/04; A63B 21/065

(52) U.S. Cl. CPC A61F 5/028; A63B 21/065; A61N 1/0484

(57) ABSTRACT

A wearable orthotic appliance to which at least one corrective element for correcting neuromuscular imbalances may be removably attached is made of fabric assembly held in compressive tension when worn by a user, the fabric assembly including a cavity or chamber into which the corrective element may be placed and secured at any of a plurality of continuously-adjustable positions. Maintenance of the corrective element position during use of the appliance may be assisted by establishing a compression gradient over selected areas of the appliance during assembly. The wearable orthotic appliance may be a shirt, vest, body suit, or other garment or article of clothing, and the corrective elements may be weights, electrodes, or other discrete elements whose therapeutic effect depends on proper positioning relative to the wearer's torso or other body parts.

### CROSS REFERENCE TO RELATED APPLICATIONS

This patent application claims priority as a continuation-in-part to U.S. patent application Ser. No. 15/425,602, filed on Feb. 6, 2017, titled “ORTHOTIC APPLIANCE WITH CONTINUOUSLY ADJUSTABLE POSITIONING OF CORRECTIVE ELEMENTS,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Described herein are wearable orthotic appliances to which one or more corrective elements for correcting neuromuscular imbalances may be removably attached at therapeutically effective locations. The wearable orthotic appliance may be a shirt, vest, body suit, or other garment or article of clothing, and the corrective elements may be weights, electrodes, vibration-causing transducers, or other discrete elements whose therapeutic effect depends on proper positioning relative to the wearer's torso or other body parts.

These apparatuses (e.g., garments, devices, etc.) and method described herein also relate to a wearable orthotic appliances in which corrective elements have been positioned by a process involving a clinician applying stimuli to an individual, observing individual reaction to the stimuli, positioning at least one weight in a cavity provided in the appliance, again applying the stimuli to the individual and observing the individual's reaction, and adjusting a position of the at least one weight within the cavity.

U.S. Pat. Nos. 7,708,673 and 7,156,792, both assigned to Motion Therapeutics, Inc. and incorporated herein by reference in their entirety, describe orthotic appliances in the form of garments that are worn on the upper body and that include pockets or slits into which weights may be inserted. The resulting weighted garments are used to improve imbalance, instability, and/or rotational asymmetry, including but not limited to loss of balance in sitting or standing and difficulties in locomotion caused by neurological disorders such as multiple sclerosis, stroke, traumatic brain injury, Parkinson's disease, injuries, and congenital conditions. The weighted garments can also be used to assist patients with orthopedic injuries causing imbalance, as well as to address balance, stability, and symmetry issues not caused by injury or disorder, in order to improve performance in athletics or daily activities.

The present invention relates generally to orthotic appliances of the type disclosed in U.S. Pat. Nos. 7,708,673 and 7,156,792, but which have an improved construction that provides easier and more accurate, continuously adjustable positioning of the weights or other corrective elements, including within targeted zones or regions. The improved construction includes replacement of weight holding pockets with panels that enable a therapist or user to continuously adjust the positions of one or more corrective elements, using differential compressive tension to maintain positioning when the appliance is worn without the need for additional clinches or straps. The result is a lightweight, comfortable, orthotic appliance with enhanced ease-of-use and improved therapeutic effects.

By way of background, further description of the manner in which garment weighting or electrical stimulation (BBTW®) weighted garment technology describe on the Motion Therapeutics website. Further background including descriptions of other uses for weighted garments, biomechanical suits, and the like, for both orthotics and athletics, may be found, by way of example, in U.S. Pat. Nos. 4,268,917; 4,658,442; 4,602,387; 5,799,328; 5,810,699; 5,957,873.

An example of a garment that combines compression and weights is found in U.S. Pat. No. 8,944,974. This patent, however, does not provide for continuous adjustment of weight positions, but rather utilizes pockets to hold individual weights at discrete positions. Furthermore, the '974 patent describes a unitary construction that is incapable of providing differential compression. While differential compression is provided in some embodiments, the differential compression is achieved by clinches.

An example of a garment that uses differential compressive tension to provide proprioceptive feedback to a wearer during movement is found in U.S. Pat. No. 9,445,932. The differential compressive tension is applied by tensor bands that are stitched into the garment, the material of the tensor bands being stiffer than the material used to make the remainder of the garment. However, U.S. Pat. No. 9,445,932 does not apply the differential compression to help secure corrective elements, including within targeted zones or stabilize the weight in three planes in response to patient movement, and there is no disclosure of achieving the compressive tension during manufacture by subjecting panels to different tensile loads as they are stitched together, rather than by using material of different stiffness. As a result, the proprioceptive feedback provided by the garment disclosed in U.S. Pat. No. 9,445,932 cannot be adjusted by a clinician or user to compensate for specific individual neuromuscular deficiencies. Also, in the garment of U.S. Pat. No. 9,445,932, a compression gradient can only be established between the bands and surrounding material, rather than continuously across and within different panels of the same material.

### SUMMARY OF THE DISCLOSURE

The present invention relates to wearable orthotic appliance to which at least one corrective element for correcting neuromuscular imbalances may be removably attached is made of fabric assembly held in compressive tension when worn by a user, the fabric assembly including a cavity or chamber into which the corrective element may be placed and secured at any of a plurality of continuously-adjustable positions (including within targeted zones or regions).

The placement of the corrective elements on a wearable orthotic appliance (e.g., garment) may be an iterative process that involves initial perturbation testing, placement of the corrective elements, further perturbation testing to evaluate the effects of the corrective element placement, adjustment of the corrective element positions, and repetition of the testing and adjustment steps until an acceptable therapeutic result is achieved. The tests may, for example, involve observing the reaction of an individual to applied linear and torsional forces in various directions while the individual is attempting to maintain a standing position, in order to determine how stable, the individual is, or how quickly the individual responds to the applied linear and torsional forces. As each stage of testing is completed, the clinician must position weights or other corrective elements within the garment so as to correct for the imbalances exposed by the testing. In some of the examples described herein, placement of the weight receiving pockets may be determined based on anatomic regions of the body. These regions may be chosen and specifically selected to improve patient balance, for example. Pockets may be placed so that if an imbalance about the body is identified, modification of force vectors can be instantaneously obtained through stimulation of the muscle imbalance in the desired force vector. Modification of anterior and posterior force vectors can be instantaneously obtained through stimulation of the muscle imbalance in the desired force vector showing imbalance based on perturbation testing. By providing for continuous positioning of the corrective elements, i.e., positioning of the corrective elements at any location within therapeutically significant areas of the garments, rather than requiring placement in discrete pockets, the clinician can more accurately position the corrective elements for better therapeutic effect. Positioning corrective elements within the targeted locations may be made in a manner that allows the position to be modified. When determining the locations to weight by testing and retesting, only areas that respond to the test and retest procedure may be weighted, in order to keep the overall weight of the garment to a minimum. In general, the corrective elements are removed from garment for laundering. The garments may be adapted to allow easy and accurate marking of the weighting location while even when laundering. None of the current pocket-based or orthotic appliances or compression garments is capable of enabling continuous positioning of corrective elements in a clinical setting, maintaining the positioning during use of the appliance outside the clinical setting, and yet of providing sufficient accessibility to enable a user to mark and thereby recreate the original positioning when the corrective elements are removed from the appliance outside the clinical setting (it should be appreciated that the term “clinical setting” in this context refers to any setting in which the positions of the corrective elements are established for therapeutic, athletic training, or other purposes).

Maintenance of the corrective element position during use of the appliance is preferably assisted by establishing a compression gradient over selected areas of the appliance during assembly.

The wearable orthotic appliance may be a shirt, vest, body suit, girdle, belt, or other garment or article of clothing, and the corrective elements may be weights, electrodes, vibration-causing transducers, or other discrete elements whose therapeutic effect depends on proper positioning relative to the wearer's torso or other body parts. The appliance itself is preferably made of a lightweight synthetic fabric material having elastic memory properties, such as Lycra®, Spandex®, a Lycra®-Spandex® blend, Neoprene®, or the like.

The orthotic appliance of the preferred embodiments is made up of a plurality of fabric panels, at least one of which includes a plurality of layers that form a corrective element positioning cavity or chamber, the corrective element positioning cavity or chamber including a material layer, coating, or other structure to which the corrective element may be removably secured, for example a layer to which an elastic Velcro® hook and loop fastener material may be removable adhered. The attachment layer is secured to at least one of an inner layer that defines one side of the cavity or chamber and an outer layer that define the second side of the cavity or chamber, the outer layer preferably forming an exterior layer of the panel. The attachment layer, inner layer, and outer layer are preferably assembled together to form a panel, which is then assembled to at least one other panel or fabric piece made of a flexible material to form the appliance.

It will be appreciated that, although a particular of arrangement of “layers” is described herein, layers may be combined or added while still achieving a desired compressive tension effect in both the panels that receive the corrective elements and the non-corrective-element receiving panels to which they are assembled, and further that the term “layers” may encompass both single and multiple ply addition of tension by differential stretching of the panels as they are assembled together, enables a compression gradient to be achieved without the need to vary the stiffness of the panels using different materials. By increasing the tension in areas where the corrective elements are to be attached, compression is increased in those areas to assist in maintaining the position of the corrective elements when the orthotic appliance is worn by a user during ordinary and athletic activities. Differential compression may also be used to ensure a proper fit and to enhance the comfort of the user.

The use of a panel construction also enables ventilation panels to be included in the appliance, in order to provide increased heat dissipation. The ventilation panels may, for example, be made of a mesh material and provided at the sides of the appliance, adjacent the wearer's armpits or any other area of the torso not subject to stimulation by the corrective elements.

In the embodiment where the orthotic appliance is an upper body garment such as a shirt or vest, the garment may be tailored to have an added length to allow the free end at a lower side of the garment to be doubled over, increasing elastic retention of the weights. In some examples, it may be beneficial to have the openings in to the chambers formed by the panel(s), or at least some of the panels, be on the bottom (lower side) of the panel when worn; in such examples it may be further beneficial to have the opening be configured to be covered by another region or panel (e.g., the added length 10 in FIGS. 1 and 2), which may add additional security.

In a preferred embodiment of the invention, the garment includes three cavities or chambers arranged to receive weights at positions most likely to provide therapeutic or performance benefits. The first is situated at the front of the garment and opening downwardly to cover the abdominal musculature, and the second and third cavities or chambers being situated on the back of the garment, one opening at the neck and the other at the bottom.

The corrective elements may be elastomeric weights embedded with metal, although other types of weights may be substituted without departing from the scope of the invention. The weights may be integrated with electrodes for causing electrical stimuli or vibration-causing transducers, the electrodes or vibration-causing transducers may be placed elsewhere on the garment, or the electrodes may be omitted. In any of the corrective elements a sensor may be included. For example, a sensor may be combined with a weight. The sensor may be a position and/or vibration sensor (such as an accelerometer or the like). Other sensors may include galvanic skin sensors, temperature sensors, etc.). the sensor(s) may transmit, including wirelessly transmit, data to a remote processor for processing, including to a handheld processor such as a smartphone, tablet, etc. that may be running software and/or firmware for receiving, storing, processing and/or transmitting the data to another remote site (e.g., cloud server) for later access, such as by a physician or therapist.

In the case of embodiments in which the corrective elements are weights, the relatively distributed weight displacement of the embodiment enables electrodes or vibration causing transducers to be freely located at positions outside the cavities or chambers.

The corrective elements are added to the orthotic appliance during an iterative process in which, following initial visual observation or perturbation testing, at least one corrective element is placed within a cavity or chamber of the appliance, further perturbation testing is carried out to evaluate the effects of the corrective element placement, the position of the corrective element within the cavity or chamber is adjusted without having to remove the corrective element entirely from the cavity or chamber and place it in a different cavity or chamber, and the testing and adjustment steps are repeated until an acceptable therapeutic result is achieved. The tests may, for example, involve observing the reaction of an individual to applied linear and torsional forces in various directions while the individual is attempting to maintain a sitting or standing position, in order to determine how stable the individual is, or how quickly the individual responds to the applied forces. The tests may also be applied to individuals in a seated position. The testing may also be performed while standing on a less stable surface (e.g., on foam) or on one foot in order to identify directional instability as indicated.

Any of the apparatuses described herein may include one or more pulls or straps that are configured to apply force to the user's body, often in an asymmetric (relative to the axis of symmetry of the body, such as the anterior-posterior axis of the body) manner. Although the examples shown herein may be of particular use for treating spinal (e.g., scoliosis and kyphosis) patient's they are not limited to these examples. Any of these devices and methods may include the use of a strap/pull as described herein. In particular, these straps and/or pulls may be configured to include attachment regions at either ends, such as a Velcro™ (e.g., hook and latch) material that may be secured within the chambers or pockets as described herein. These straps/pulls may pass through one or more slots or slits in the panels of these garments/orthotics. For example, a first end of a strap/pull may be attached by the user (e.g., therapist, clinician, etc.) within a chamber or region of the panel and pass out of the panel into a second pocket or chamber in a second panel. The strap/pull may pass through one or more slots in each of the panels.

In any of these examples a strap/pull may be included as part of a tether system to hold the straps/pulls as described herein. For example, a strap or pull may engage with a guide, slot or channel in the garment/orthotic to help secure and/or direct the force of the strap/pull.

For example, described herein are wearable orthotic appliances configured to releasably attach to at least one corrective element for correcting neuromuscular imbalances that include: an assembly made up of a plurality of stitched-together panels of resilient fabric wherein adjacent panels are stitched to provide differential compressive tension against a user's torso when worn, the assembly including a plurality of chambers configured to receive and secure the at least one corrective element at any positions within each of the chambers; wherein the chambers are located on the panels applying the higher compressive tension and are present on each of: an abdominal region, an upper back region and a lower back region; a plurality of slits in the stitched-together panels configured to provide access for an end of one or more straps into the chambers of the panels; a bottom panel configured to fold over an opening along a bottom of the lower back region panel to increase compression over the wearer's lower torso; and a plurality of straps having ends configured to be secured within the chambers. In any of these examples the strap(s), e.g., the plurality of straps, may be configured and arranged on the garment/orthotic to provide a rotational pull. In any of these apparatuses and methods the straps/pulls may be arranged or attached asymmetrically relative to the patient's axis of symmetry.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a front view of a weighted garment constructed in accordance with the principles of a preferred embodiment of the invention.

FIG. 2 is a back view of the weighted garment of FIG. 1.

FIG. 3 is a cross-sectional view of the weighted garment of FIG. 1.

FIG. 4 is an example of a garment configured for treating a patient having curvature of the spine.

FIG. 5 is an example of a panel for treating a patient having curvature of the spine.

FIG. 6 is an example of a panel for treating a patient having curvature of the spine.

FIGS. 7A-7D illustrate examples placements of weights to treat different spinal curvatures (e.g., thoracic, thoracolumar, lumbar, and double major curve, respectively).

FIGS. 8A-8D schematically illustrate examples of the placement of straps to apply force in addition to weight placement to treat different spinal curvatures (e.g., thoracic, thoracolumar, lumbar, and double major curve, respectively). In this example, FIGS. 8A, 8B, and 8D illustrate the use of one or more, including double, pulls (e.g., straps or pull straps) that may apply additional focal force in addition to supporting and/or assisting the corrective element(s).

FIG. 9A shows a front view of another example of a garment as described herein.

FIG. 9B shows an example of a back view of a garment as described herein.

FIG. 10A shows a front view of another example of a garment as described herein.

FIG. 10B shows an example of a back view of a garment as described herein.

### DETAILED DESCRIPTION

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

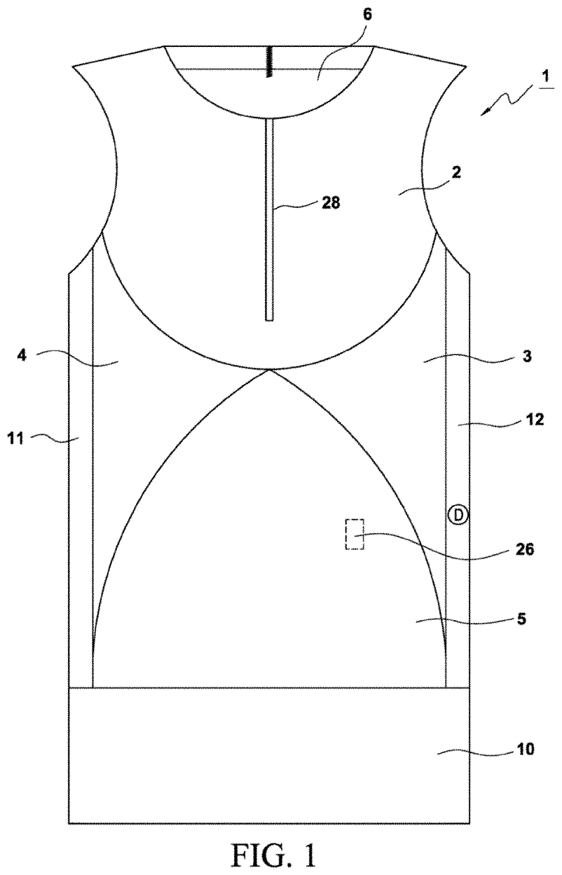

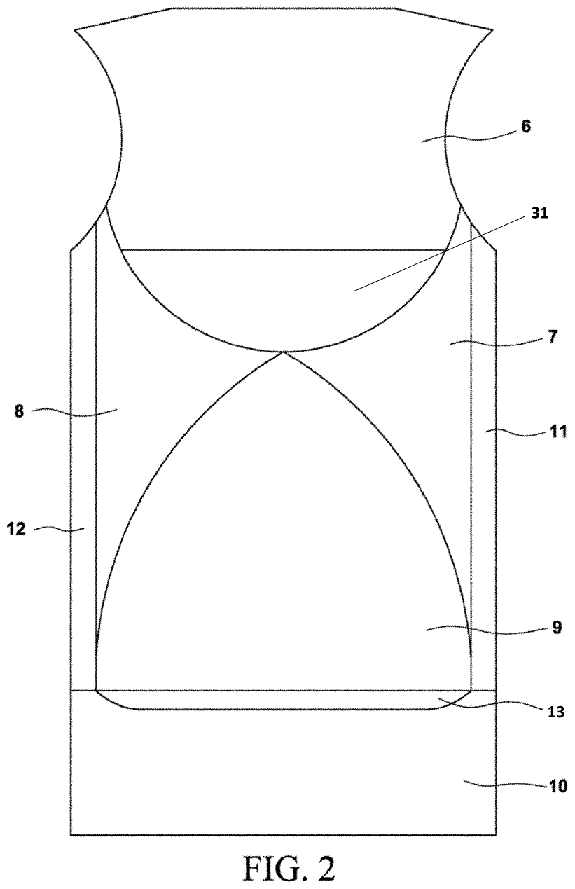

Although the present invention applies generally to an orthotic appliance, a preferred embodiment of the invention is in the form of a garment such as a vest or shirt. As illustrated in FIGS. 1 and 2, the weighted garment 1 constructed in accordance with the principles of a preferred embodiment of the invention is made of a light weight, breathable elastomeric material. The material may be a Lycra®, Spandex®, or Lycra®-Spandex® material, or another lightweight material having elastic memory properties, such as Neoprene®. When assembled into a garment, the garment is intended to fit snugly against the user's torso.

According to the preferred embodiment, the garment is constructed to exert a compressive force against the user's torso when the garment is worn. The compressive effect may be uniform or differential, i.e., the compressive force may vary at different locations across the garment. For example, a differential compressive effect may be used to apply a greater compression in areas where the corrective elements are to be positioned, and a lower compression in other areas. A differential compressive effect may be the result of different panel shapes, may be achieved by assembling panels of the garment while under tension, whether uniform or varying, or by combinations of panel shape effects and the application of uniform or varying tension.

As shown in FIGS. 1 and 2, the front and back sides of the garment may be constructed of a plurality of solid panels 2-10, mesh panels 11 and 12, a foldable lower panel 13 that extends around both the front and back of the garment, each made of a resilient breathable material as described above, and an optional back crease panel for added stability. The various panels are attached to each other at seams 12-20 having suitable resilience and strength to hold the panels together when under tension, for example by flat lock seam stitches or any other stitches or other material joining arrangements of techniques capable of joining panels of flexible material.

In the illustrated embodiment, three of the panels 8-10 are made up of multiple layers to form cavities or chambers into which corrective elements in the form of weights 26, electrodes (not shown), vibration-causing transducers (not shown), or other corrective elements may be fixed or removably inserted at varying positions within the chambers of cavities. It is preferred that the areas within the cavities or chambers to which the corrective elements are attached be large enough to cover an entire range of positions over which a corrective element may be adjusted after initial positioning, and that a panel be provided for each therapeutically significant area of the individual's torso. As a result, the cavities or chambers have a width that is a multiple (for example, 1.5, 2, 2.5, or 3) of the corresponding dimension of one of the corrective elements and extends over an entire region of the user's or individual's torso. In the illustrated embodiment, these regions include an area at the upper back 6, an area at the lower back 9, and an area in the front of the torso, with each area extending to both the right and left of the individual's vertical center or spine, although it is also possible to provide separate cavities or chambers on each side of the spine.

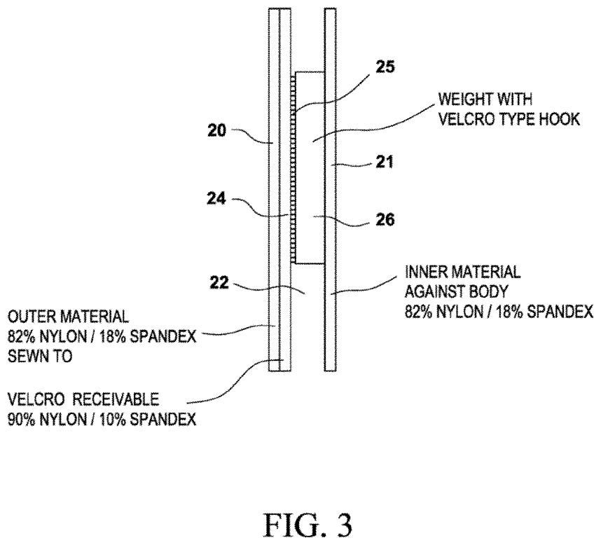

As shown in FIG. 3, the cavities or chambers may, for example, be formed of two 82% nylon/18% Spandex® power knit layers 20, 21 that are attached to the interior side of the respective panels, with the cavity or chamber 22 being formed between the nylon/Spandex® layers 20 and 21, and a Velcro® attracting or fixing layer 24 made up of 90% nylon/10% Spandex® added to an interior side of one of the nylon/Spandex® layers, for example nylon/Spandex® layer 20, as illustrated in FIG. 3. The Velcro attracting layer 24 attracts a corresponding Velcro® layer 25 on the weight 26 when the weight is positioned in the cavity or chamber 22. The three layers 20, 21, and 24 may be secured to each other and to the outer panels by any suitable method, including conventional stitching on three sides of each piece with the fourth side left open, and may further include closures or flaps for the openings. The lower back and front corrective element receiving panels 5, 8, and 9 may, in the illustrated example, be further secured by folding of the bottom panel 10 over the lower openings, which also has the effect of increasing compression over the individual's lower torso.

Although a three-layer construction is shown, the invention is not limited to three layers or to the illustrated materials. For example, either the outer layer or the inner layer may itself be capable of holding the corrective elements, by including adhesive, magnetic, or mechanical fixing properties, and therefore the separate layer 24 may be omitted. Alternatively, additional layers may be added for any purpose, including structural reinforcement, perspiration removal, anti-microbial effects, or sensing of corrective element positions.

The shapes of the panels help determine the compressive force distribution across the garment and also the areas to which the corrective elements may be affixed. In the illustrated embodiment, corrective element receiving panels 7 and 8 (and 3 and 4 on the front of the garment) have a generally triangular shape that extends approximately midway up the garment and is widest at the base of triangle. The panel 6 at the upper rear of the garment has a substantially rectangular shape, with upper and side curvatures to accommodate arm and neck shapes, and a slot for facilitating fitting of the garment over the user's head, and a closure such as a zipper 28, while the solid panel 2 at the upper front has a shape that accommodates a user's chest, arms, and neck. Hyperbolically triangular panels 3, 4, 7 and 8, rectangular side panels 11 and 12, and the continuous belt-like panel 10 complete the front and back sides of the garment. Those skilled in the art will appreciate that these shapes are described by way of example only, and not intended to limit the overall scope of the invention.

The corrective elements to be placed in the cavities or chambers may be conventional weights, such as parallel piped elastomeric members into which metal has been embedded and that are available in units of 1/16, ⅛, ¼ or ½ pound, although it will be appreciated that the weights, dimensions, shapes, and composition of the weights may be varied without departing from the scope of the invention. Suitable electrode pads may include integrated power sources and be activated wirelessly or connected by wires to a central controller and/or power source.

A process by which the corrective elements may be placed in the garment is to assemble a weighted orthotic appliance is generally described in U.S. Pat. Nos. 7,156,792 and 7,708,673. In general, the process is an iterative process that involves initial perturbation testing, placement of the corrective elements, further perturbation testing to evaluate the effects of the corrective element placement, adjustment of the corrective element positions, and repetition of the testing and adjustment steps until an acceptable therapeutic result is achieved. The testing steps involve observing the reaction of an individual to linear and torsional forces applied by the clinician in the form of pushes in various directions while the individual is attempting to maintain a standing position, in order to test the individual's stability or how quickly the individual responds to the applied forces. As each stage of testing is completed, the clinician positions one or more weights or other corrective elements within the cavities or chambers. By providing for continuous positioning of the corrective elements, i.e., positioning of the corrective elements at any arbitrary location within the cavities or chambers, rather than requiring placement in discrete pockets having a limited extent, the clinician can more easily and accurately adjust the position of the corrective elements for better therapeutic effect, without the need to remove the corrective elements from the cavities or chambers. Furthermore, by making the cavities or chambers more easily accessible, a user of the appliance can mark positions of the corrective elements within the cavities or chambers so as to more easily reposition the corrective elements after removal for laundering or dry cleaning.

It will be appreciated by those skilled in the art that, although the above-described process and appliance made by the process is very useful for the treatment of neuromuscular disorders, the invention is not limited to garments having corrective elements placed using the specific procedure described above. Instead, the differential compressive tension of the preferred embodiment assists in maintaining corrective element positioning for corrective elements that have been placed by any procedure, while the cavities or chambers providing in selected panels of the garment facilitate positioning of the corrective elements at desired locations on either side of the spine and/or torso by any position-determining procedure, whether involving stimulus application and observation of individual reactions by a clinician, followed by retesting and adjustment, or any other manual or automated test, evaluation, and position-determining or any other analytical calculating methods.

The side panels 11 and 12 may be attached to the solid fabric panels 1-10 by the same flat lock stitched seams used to hold attach the solid fabric panels to each other, or by any other fabric panel attachment means. The material of the mesh panels may be the same as the other panels, but the mesh panels are arranged to facilitate heat dissipation by increased ventilation. Although provided in the form of side panels positioned under the user's arms, the mesh panels 11 and 12 may alternatively, or in addition, be provided at other positions on the front or back of the garment or may be omitted replaced by cut outs or inserts in the other noncorrective element attachment panels.

Finally, the illustrated embodiment may include a back crease to allow relative movement between the upper and lower back panels to be optionally provided for extra stability.

### Garments for Treating Scoliosis

Scoliosis and kyphosis (and kyphoscoliosis) are spinal deformities that are caused by inadequate muscle strength and tightness in some areas from unequal pulls of the muscles, driven by signals in the neuromuscular system. Kyphosis is generally seen in the thoracic area however could also be noted as a reverse lordosis in the lumbar spine. Scoliosis can be seen throughout various regions the spine. Irregular posture is often a consequence. In addition, these disorders can cause difficulty with breathing, digestion, balance, confidence and appearance. Providing an undergarment or shirt that can enable improved facilitation to the muscles and structures underneath is valuable and novel in its form.

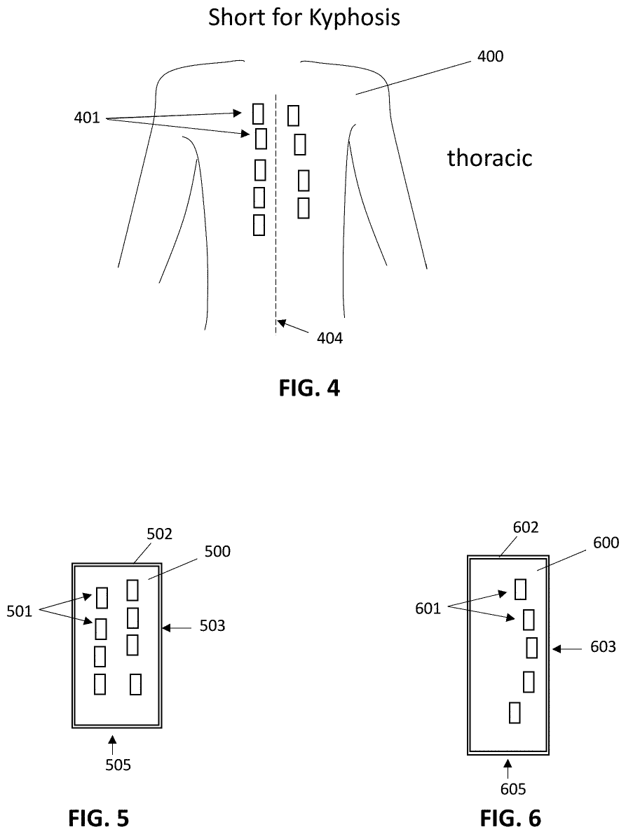

The garments described herein may be used to treat scoliosis and/or kyphosis. In some variations the garments described herein may also or alternatively include bracing, such as an insert for a flexible stay and/or moldable metal or plastic. For example, FIG. 4 illustrates an apparatus for improving or stabilizing scoliosis. In the variation shown in FIG. 4, the garment (e.g., shirt) includes an inside panel that is configured to allow weight placement as drawn for various patterns of scoliosis (or in some examples, kyphosis). In some examples the corrective element(s) may be positioned adjacent to the spine on one (scoliosis) or both (kyphosis) sides. For example, the corrective elements may be weights placed on the right side of the spine (axis of symmetry 404) in FIG. 4. The panel include a pocket that may open on one lateral side, but be closed on the other lateral sides, or may be open on the bottom, but closed on the top, over the back of the patient, e.g., along the spinal region. Light weights (e.g., 1/16^{th}-⅛^{th} pound increments) may be placed on the garment within this pocket, so that the weight (and/or stimulator) may be worn by the garment between the transverse process and the spinous process at on the convex side of the vertebrae to activate the spinal rotator, extensors and lateral flexors. Weights may also be placed further laterally at the level of the convex curvature on muscles such as the Iliocostalis. Weights can be placed anywhere along the curvature of the spine in the shirt. In some examples a midline region of the garment (shown in FIG. 4 by the dashed line) may be marked or the back region may be divided up into two separate regions separated by the midline. When the user wears the garment, the midline may be aligned by the caregiver or medical user (e.g., therapist, physician, nurse, etc.). An additional compressive pull at the apex of the spine can be added and an elongation pull to the concave side of the curvature in opposing directions. FIGS. 5-6 show examples of additive panels that may be used as described herein, particularly (but not exclusively) where the user can add the corrective elements (e.g., weights) so that they are then attached to the shirt, allowing for maximal adjustment of individuals sizes and curvatures. In FIGS. 5 and 6, the panel may be a separate panel that may be part of any of the garments described herein. For example, the panel may be included in place of (or in addition to) panel 6, 31, or 9 shown in FIG. 2. Alternatively, the panel may be attached to a garment, including an orthotic or brace.

In some examples, the corrective element may be a weight that is long and thin. For example, a long (e.g., between 1-12 inches, between 1-6 inches, between 2-10 inches, between 3-12 inches, between 1-3 inches, between 2-4 inches, between 2-5 inches, etc.) and thin (and in some cases, narrow, e.g., between 0.1 inches-1 inch, between 0.1 inches-0.5 inches, etc.) weighing between 1/16 to ½ lb. (e.g., between 1/16-¼ lb., etc.) may be applied along the spine, either at the midline region, on both side so the midline, or on one side, including along the length of the curvature. The long thin weight may be flexible, including flexible.

In FIG. 5 the panel may be attached to shirt in any appropriate manner and may be configured for treating kyphosis. In FIG. 5, the panel may be formed of one or more layers 502 as described above. The panel may form a pocket having an opening on the side 503 or bottom 505, and may include a surface configured, as described above, to hold one or more corrective elements (e.g., weights, including in particular, light weights) 501. FIG. 6 shows another example of a panel, which may be configured for treating scoliosis. As in FIG. 5, the panel may be formed of one or more layers 602. The panel 600 may form a pocket having an opening on the side 603 or bottom 605, and may include a surface configured, as described above, to hold one or more corrective elements (e.g., weights, including in particular, light weights) 601.

The panels described herein may also be configured to be added to an existing orthotic and/or garment. For example, a panel such as that shown in FIG. 5 or 6 may be added to a brace or garment (including, but not limited to a tightly fitting garment, such as a garment made from an elastic, form-fitting material). The panel may be permanently and/or removably attached. For example, the panel may be configured to be attached to the garnet by a fastener, such as a hook (pins), attachment (snap, button, etc.), loop-and-hook) e.g., Velcro™) or the like. The patch may be removed and washed or applied to another garment and/or orthotic, including a brace, such as a spine orthotic or orthosis).

Any of the garments described herein may also be configured to apply force to de-rotate the spine. For example, any of these garments may include one or more straps (e.g., elastic straps) pulled diagonally from the shoulder around the ribs to pull laterally and to de-rotate the spine. In some examples, the garment may include one or more slits in the shirt at a side or woven through an inside attachment for a desired attachment configured to be positioned at a level of the convex curvature of a patient.

In these examples, because of the overlapping shirt regions at the bottom of the garment, the patient may not need to have attachment around the legs. Thus, these garments may include one or more light weights placed on specific muscles to stabilize the spine and may include one or more compressive pulls on the convex curvature and/or one or more elongation pulls to the concave muscles. For example, elastic diagonal pulls may also provide stabilization to the weighted panel. The weights can be on a panel and attached by latch-and-hook attachment (as described herein) into the shirt or within the shirt. In any of these examples, the weights may be made of a soft, flexible material and may weigh between 1/32 and ½ pounds, and may be very thin (e.g., 3 cm thick or less, 2 cm thick or less, 1.5 cm thick or less, 1 cm thick or less, 9 mm thick or less, 8 mm thick or less, 7 mm thick or less, 6 mm thick or less, 5 mm thick or less, etc.).

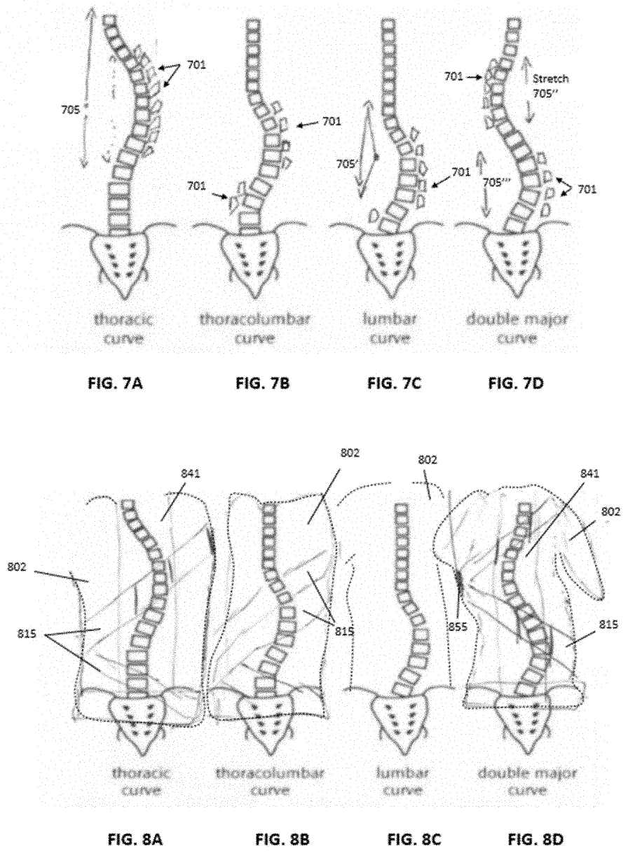

FIGS. 7A-7D illustrate examples of spacing of weights to treat the spinal curvatures indicated in each figure. In FIG. 7A, the thoracic curvature that may be treated by including a series of low weights 701 (e.g., between 1/32 pounds and 1 pound, between 1/32 and ½ pound, between 1/16 and 1 pound, between 1/16 and ½ pound, etc., where in some examples lighter is better) that may be arranged on unilaterally on the side of the spine in the direction of the curve, as shown. The garment may be configured to apply a pulling force as indicated by the arrows 705. FIG. 7B shows an exemplary placement of weights 701 for treating a thoracolumbar curvature, configured to apply force by the application of the relatively light weights arranged down the spine as shown, held within the spinal region of the garment.

FIG. 7C shows an example of weights placed on a garment as described herein for treatment of lumbar curvature. The weights 701 may be positioned on the garment to apply forces in the directions indicated 705′ by virtue of the garment allowing them to be positioned and held securely against the body in the region of the body shown, when the garment is worn.

FIG. 7D shows an example of placement of weights on a garment as described herein for treatment of a double major curvature. In FIG. 7D, the combination of weights may provide force in the directions shown 705″, 705′″.

Any of the garments described above, may be specifically adapted for treating scoliosis and/or kyphosis. In some examples, the garment may include one or more panels along the spinal regions shown, so that the spinal panels (similar to panel 9 and or panels 9 and 31), including panels such as those shown in FIGS. 5 and 6. In any of these examples, the garment/orthotic may also be configured to apply force, including by applying directed compressive forces. The compressive force may be directed unilaterally, e.g., from a shoulder region toward one side of the spine. The compressive force may be applied via the elastic material forming the layers, and/or by the attachments between the layers. Alternatively or additionally, the force may be applied by one or more elastic straps that are integrated into the garment.

FIGS. 8A-8D illustrate schematically where straps applying force, which may be used in combination with a weighting scheme as illustrated in FIGS. 7A-7D, above. In FIGS. 8A-8D a garment 802 is shown schematically over each representation of the spine, and one or more straps 815 are shown as they be poisoned on the garment to assist in providing force (as shown in FIGS. 7A-7D) including in combination with weights positioned on the garment. For example, FIG. 8A shows an example of the position of a compressive force (e.g., applied by one or more straps and/or the configuration of the garment itself when forming the panels under a compressive load, as described above) for treating a thoracic curvature of the spine. FIG. 8B shows a configuration for treatment of thoracolumbar curvature. FIG. 8C shows an example without a strap or pull. FIG. 8D shows a configuration for treatment of a double major curvature. In any of these examples the garment/orthotic may include a support to which the pull (e.g., strap) may be attached, adjusted and/or fastened. For example, a piece of plastic may be used to reinforce or secure the strap (e.g., pull) against the outside of the body, such as a region of the body where the pulls for most of the curvature are positioned. These reinforcements may be positioned on a front of the body, and may pull the anatomy (e.g., ribs) down where necessary, e.g., to correct a rib flare. For example, a corrective pull could be applied through an addition plastic support on the side of the curve 855 (on a side of garment).

For example, in some variations the garments described herein may include one or more straps that may be applied to the Velcro within the pockets (or otherwise on the garments) in the patterns shown in FIGS. 8A-8D. In FIGS. 8A and 8D the strap(s) may be on the back; in FIG. 8B the strap(s) may be on the front of the garment. In some variations the straps may be included in slots or channels formed in the garment. In some examples the pockets may include one or more slits or openings to allow the straps to be secured within the pocket and pass out of the pocket over a region of the shirt and back into another portion of the same pocket or a different pocket where they may be attached on the other end of the strap.

For example the apparatuses described herein may be used to treat Kyphosis. These apparatuses may be used as part of a brace, including a rigid brace (or a brace including rigid portions that may be coupled to the patient by one or more straps. Thus, these apparatuses may be configured to include a panel for attachment of weights as described above (e.g., in FIGS. 5-6). The garment may be configured so that the weights are placed parallel to the spine at specific vertebrae to enhance spinal extensor facilitation with the light weights. By spreading out the weight in small increments it may decrease the load in any particular are which decreases the possibility of fracture in osteoporotic spines. Optionally, the garment may include a panel in the front which can be included to receive weights to counter the asymmetrical forces pulling posterior.

In any of these apparatuses (e.g., garments/orthotics) the apparatus may include a longitudinal or vertical placement of a stretchy unbroken loop (UBL) fabric 841 that may be attached (e.g., sewn) straight down the spine between up to 5 inches wide depending on the level of scoliosis to cover. As illustrated in FIGS. 7A-7D, weights (e.g., rectangular, lightweight, 1/16th and-⅛th pound weights) may be positioned at each segment of the spine, placed on the convex side of the curvature to facilitate the spinal extensors and rotators at the thoracic and lumbar curvature. In some examples, a vertical strap optionally be used to pull vertical in opposite directions to provide a stretch on the concave portion of the spine. In some examples a de-rotational strapping may be placed through a slit on the side of the garment or on the outside of the garment as mentioned above.

Any of these examples may include a longitudinal or vertical panel (e.g., of or including a stretchy unbroken loop fabric) attached straight down the spine. For example, flexible metal or plastic stays may be sewn into the UBL. Small weights (e.g., between 1 1/16th and ⅛th pounds) may be placed adjacent to the spine bilaterally to facilitate segmental extension. Additional panels may be placed in the abdominal cavity to counter the posterior loss of balance from the weight placement. In some examples, the apparatus may include an elongated flexible weight positioned, e.g., 6-8 inches apart, distributing up to about between about ¼ pound to ⅔ pounds; the weight could have Velcro and/or silicon to position in or on the shirt as described herein.

The apparatuses described herein may include one or more pockets (e.g., cavities) into which corrective elements (e.g., weights) may be applied. These pockets serve as windows to access each individual patient's muscle imbalance, using corrective elements (e.g., weight and/or electrical stimulation) to facilitate the inhibited system. These apparatuses may allow for unique anatomical alignment and integration, and therefore individuals weighting patterns. Areas on the garment (e.g., shirt) that are not considered targeted locations for needed increased neuromuscular stimulation in order to increase stability may not include a pocket and thus do not have Velcro receivable fabric panels, allowing for a lighter weight system. These pockets may serve as windows to access each individual patient's muscle imbalance, using corrective elements to facilitate the inhibited system. Further, the compressive nature of the panels and therefore these garments may further stabilize the weight on the shirt allowing it to stay in desired location with movement including running and rotational activities.

In any of these examples, the garment may include silicone on the inside of shirt to assist with keeping shirt in desired location directly on the skin allowing for increased somatosensory facilitation. The use of silicone may also increase somatosensory facilitation in the targeted zone.

The garments described herein may be configured to modify the operation of anatomic “slings” in the body. Anatomic slings are comprised of muscle, fascia, and ligament all working together. When muscles contract, they produce a force that spreads beyond their attachments of the active muscle. These forces are transmitted through structures within an anatomic sling which helps carry the force of the muscle far from the origination of the originating contraction. This may be referred to as a force vector. Muscles within a sling are connected to other muscles via myofascia. Fascia connects structures. When a muscle is activated, force generates through the muscle into the fascia. The force then moves into the connecting muscle adjacent to the original muscle contraction. This forms the sling and force vector. Force vectors assist in transfer of loads within the trunk, lumbar spine and pelvis.

Muscles within slings overlap and connect with other slings depending on the change in force vector needed for dynamic movement and function. When balanced, anatomic slings assist in providing optimal alignment of bones and joints throughout movement. When an imbalance occurs at the trunk, imbalanced force vectors cause altered tension in the myofascial sling. This malalignment can contribute to loss of stability during static and dynamic tasks. One example sling is the anterior oblique sling (AOS). The anterior oblique sling consists of the external oblique, internal oblique, connecting contralateral adductor muscle via the adductor abdominal fascia. When this group contracts, it provides support as if it was serving as an abdominal binder and increases abdominal stability. Accelerating, decelerating, and changing speeds in sports load the AOS, and imbalance can lead to injury of the abdomen, hernia, and recurring groin injury. For example, accelerating, decelerating, and changing speeds while playing sports all load the AOS. Imbalance can lead to abdominal strain, hernia, and recurring groin injury.

Similarly, the posterior oblique sling (POS) consists of the latissimus dorsi, the gluteus medious and maximus, and the interconnecting thoracolumbar fascia. The propulsive phase of gait is highly implicated with the POS. pushing off with the leg while the trunk undergoes contralateral rotation with pelvic stabilization is achieved through this sling. The AOS and POS are sometimes considered agonist antagonist paired muscles, required to work together for smooth function. When one sling contracts in a shortening manner, the other works in a lengthening manner to control the motion. The deep longitudinal sling (DLS) is another sling group. The lateral sling (LS) connects the erector spinae, multifidus and the thoracolumbar fascia and ligaments in the pelvis. The sling allows movement in the sagittal plane while maintaining local stability. The LS consists of the gluteus medious, minimus, tfl, and ITB. It has been proposed to assist in lumbo pelvic stability. Smooth efficient movement requires dynamic interplay between the above slings to ensure static and dynamic stability.

In lower crossed syndrome, changes in the trunk may occur due to shortening of the upper muscle fibers of the trapezius muscles and levator scapula as well as the sternocleidomastoid. Deep muscle flexors of the head and neck and lower fixators of the shoulder girdle often become weak. At the same time, paravertebral muscles of thoracic spine also weaken in response to hyperactive chest, e.g., hypertonic pectoralis minor and major. Flexors of the hip may become shortened, along with erectors of the spine. This syndrome typically leads to weakened gluteal muscles. Weakening of the gluteal muscles can then lead to further core muscle changes including hyper-lordosis indicative of muscle imbalance of the core system as defined by weakening and hypertonicity of transverse abdominus, pelvic floor, and long spinal extensor muscle.

The apparatuses described herein, including pockets or cavities have the ability to hold weight and/or electrical stimulation to facilitate adjusting each individual's specific muscle imbalance. The targeted sensory inputs (e.g., weight or vibration, or electrical stimulation) from the corrective element(s) can serve to inhibit muscles that are too tight or overactive or facilitate muscles that are inhibited. In particular, these apparatuses may be configured so that the pockets are arranged in advantageous positions to be able to facilitate all of the potential imbalances in sling systems or in the layer syndromes.

There are ways that our bodies muscles fire that are considered optimal or efficient. When muscle imbalance or injury occurs, patients are required to alter their learned patterns of movement in order to compensate and learn a new pattern. These learned patterns can be adaptive or maladaptive. Performing mass practice in a short period of time ensures the best potential for learning new muscle firing patterns and learning of a new skill. It has been proposed that 10,000-40,000 repetitions of a task must be performed in a short period of time in order to learn a new skill. Somatosensory cues, vibration, and electrical stimulation can be used to help change muscle firing patterns. Wearing a device causing constant feedback to the imbalanced system may vastly speed up this motor learning process.

The apparatuses (e.g., garments) described herein utilize somatosensory modification in targeted areas of the trunk to elicit changes in function. The targeted receivable pockets are arranged in areas that correspond with the anterior and posterior sling, as well as the inhibited muscles in upper and lower crossed syndromes. For example, these garments are configured so that weights are able to be positioned within the pocket in order to stimulate muscles and fascia in the sling to maximize function. Using perturbation testing, increased or decreased sensory input can be utilized in the pocketed targeted areas as each individual patient requires. As less information is needed, less stimulus may be applied as the patient achieves motor learning and function is optimized.

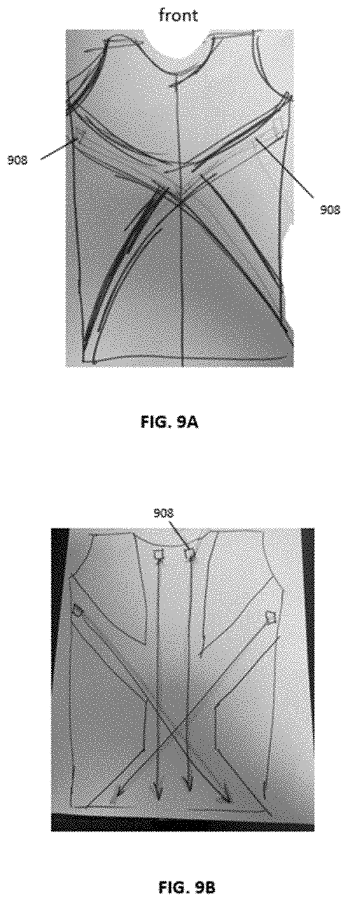

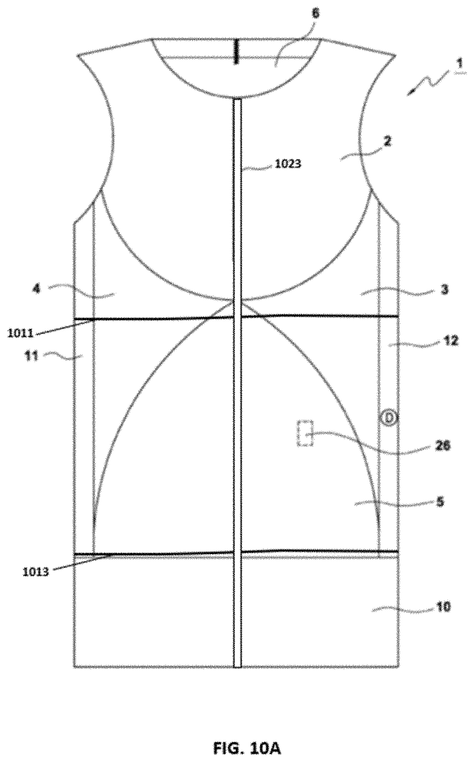

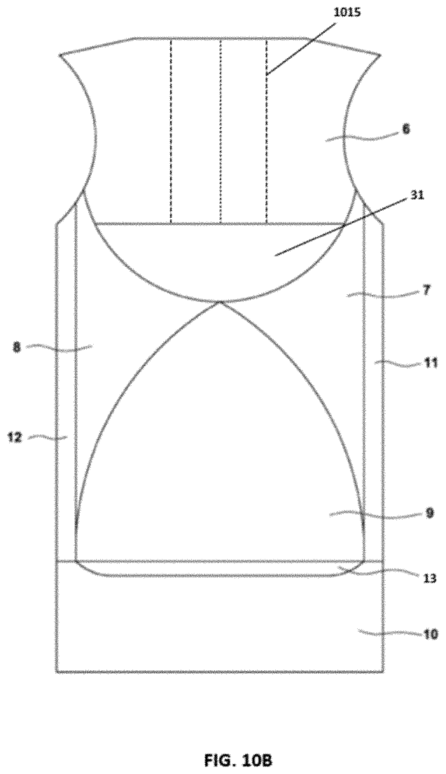

FIG. 9A illustrates another example of a garment as described herein. In the example shown in FIG. 9A, the front panel is enlarged and extended into the axilla in order to help facilitate the anterior sling. Sling assist strapping 908 can be utilized to augment somatosensory information in the pocket in addition to a corrective element (weight, electrical stimulation or vibrational stimulation) as indicated in order to achieve the desired effect. Hook and loop attachment within the cavity is used within the anterior cavity.

FIG. 9B shows an example of a cavity system that allows for sling assist strapping that follows the line of pull of the latissimus dorsi and contralateral glut maximus through the thoracolumbar fascia (mimicking the posterior sling). Th arrows in FIG. 9B illustrate the direction of force applied by the optional straps. As illustrated and described above, in reference to scoliosis and kyphosis, in some examples only one (e.g., asymmetrically applied) strap may be used, or strapping may be asymmetric.

FIG. 10A shows an example of a full zip vs ¼ zip 1023, as well as siliconized attachments 1011, 1013 below chest and at waist to prevent shirt movement and obtain accurate and consistent somatosensory cue to desired region that may be used with any of the apparatuses described herein. In this example the zipper may extend partially or completely down the length of the garment. FIG. 10B shows an example of the back of a garment as described herein, including a stretchy unbroken loop fabric region 1015 along the spine. In some examples the garment includes an elastic/siliconized seam at an interface of the pockets that have tension built in to facilitate fascial plane lines, anterior and posterior sling modification/amplification systems. In some examples, the garment includes a silicone material at a base of shirt to prevent it from riding up.

Any of these apparatuses (and methods of using them) described herein may include the use of materials having directional stretch. In general, these garments may be compression garments formed of compression fabric. The panels may be configured to create a compression gradient using either or both 4-way and 2-way stretching material. This may be configured to allow the corrective element (e.g., weight) to stay in a precise location without moving as the patient moves. In general, a stretch fabric may be either 2-way stretch or 4-way stretch. 2-way stretch fabrics stretch in one direction, usually from selvedge to selvedge (but can be in other directions depending on the knit). 4-way stretch fabrics, such as spandex, stretches in both directions, crosswise and lengthwise.

Further, although the apparatuses (e.g., garments, braces, etc.) described herein are described primarily as shirts or garments to be worn on the torso of the patient, in some examples the garments described herein may be configured to include a region that extends over the hips, buttocks, groin, and/or thigh regions. For example the garments/orthotics described herein may include a shorts or pants portion, which may help stabilize the torso portion of the apparatus. In some examples the shorts or pants may be used to anchor or attach a strap (but may not be weighted, for example). Alternatively or additionally, a strap/pull may be secured to the patient's pants or shorts, or a pair of pants or shorts adapted for coupling to the strap(s)/pull(s).

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A wearable orthotic appliance configured to releasably attach to at least one corrective element for correcting neuromuscular imbalances, comprising:
an assembly made up of a plurality of stitched-together panels of resilient fabric wherein adjacent panels are stitched to provide differential compressive tension against a user's torso when worn, the assembly including a plurality of chambers configured to receive and secure the at least one corrective element at any positions within each of the chambers;
wherein the chambers are located on the panels applying the higher compressive tension and are present on each of: an abdominal region, an upper back region and a lower back region;
wherein the lower back region comprises a bottom opening into the chamber of the lower back region panel; and
a bottom panel configured to fold over the lower opening to increase compression over a wearer's lower torso.

2. The wearable orthotic of claim 1, wherein said compressive tension includes a compression gradient that assists in maintaining a position of the at least one corrective element within the cavity or chamber.

3. The wearable orthotic of claim 1, wherein the wearable orthotic appliance is a garment or article of clothing.

4. The wearable orthotic of claim 3, wherein the garment or article of clothing is a shirt or vest.

5. The wearable orthotic of claim 1, wherein the corrective elements are weights whose therapeutic effect depends on proper positioning relative to the torso.

6. The wearable orthotic of claim 1, wherein the corrective elements include electrodes for delivering neuromuscular stimulation.

7. The wearable orthotic of claim 1, wherein the corrective elements include weights and electrodes.

8. The wearable orthotic of claim 1, wherein at least one of the panels of the plurality of stitched-together panes is constructed of a plurality of layers that form the at least one corrective element positioning cavity or chamber, the corrective element positioning cavity or chamber including a material adapted to attract and removably secure the at least one corrective element.

9. The wearable orthotic of claim 8, wherein the plurality of layers include an outside fabric layer, an inside fabric layer, and a third layer including said material adapted to attract and removably secure the at least one corrective element.

10. The wearable orthotic of claim 9, wherein the third layer is made up of a hook and loop fastener attracting material.

11. The wearable orthotic of claim 10, wherein the third layer is secured to the inner layer or the outer layer of the cavity of chamber, and the inner and outer layer are secured to each other to form a discrete panel for assembly to panels made up of a single fabric layer.

12. The wearable orthotic of claim 8, wherein the panels are assembled together under tension to generate the compressive tension.

13. The wearable orthotic of claim 12, wherein a compression gradient is introduced during manufacture by differential stretching of the panels as they are assembled together, the differential tension increasing the tension in areas where the corrective elements are to be attached to assist in maintaining the position of the corrective elements when the orthotic appliance is worn by a user.

14. The wearable orthotic of claim 8, wherein the panels include at least one ventilation panel and made of a single layer resilient mesh material.

15. The wearable orthotic of claim 1, wherein the resilient fabric is a lightweight synthetic fabric material having elastic memory properties.

16. The wearable orthotic of claim 1, wherein a number of the cavities or chambers is at least three.

17. The wearable orthotic of claim 1, wherein the abdominal region cavity or chamber includes an opening downwardly from near the user's solar plexus, and the lower back region cavity or chamber includes an opening at a lower back area of the user's torso.

18. The wearable orthotic of claim 1, further comprising an unbroken loop fabric region extending parallel to the wearer's spine on the lower back region.

19. A wearable orthotic appliance configured to releasably attach to at least one corrective element for correcting neuromuscular imbalances, comprising:
an assembly made up of a plurality of stitched-together panels of resilient fabric wherein adjacent panels are stitched to provide differential compressive tension against a user's torso when worn, the assembly including a plurality of chambers configured to receive and secure the at least one corrective element at any positions within each of the chambers;
wherein the chambers are located on the panels applying the higher compressive tension and are present on each of: an abdominal region, an upper back region and a lower back region;
a bottom panel configured to fold over an opening along a bottom of the lower back region panel to increase compression over the wearer's lower torso; and
a plurality of corrective elements.

20. A wearable orthotic appliance configured to releasably attach to at least one corrective element for correcting neuromuscular imbalances, comprising:
an assembly made up of a plurality of stitched-together panels of resilient fabric wherein adjacent panels are stitched to provide differential compressive tension against a user's torso when worn, the assembly including a plurality of chambers configured to receive and secure the at least one corrective element at any positions within each of the chambers;
wherein the chambers are located on the panels applying the higher compressive tension and are present on each of: an abdominal region, an upper back region and a lower back region;
a plurality of slits in the stitched-together panels configured to provide access for an end of one or more straps into the chambers of the panels;
a bottom panel configured to fold over an opening along a bottom of the lower back region panel to increase compression over the wearer's lower torso; and
a plurality of straps having ends configured to be secured within the chambers.
